(19) United States
(12) Patent Application Publication
(10) Pub. No.: US 2007/0129438 A1
(43) Pub. Date: Jun. 7, 2007

(54) Method for suppressing proliferation of abnormal prion protein

(76) Inventors: Yuuichi Murayama, Tsukuba-shi (JP); Masami Moriyama, Yokohama-shi (JP)

Correspondence Address: CROWELL & MORING LLP;INTELLECTUAL PROPERTY GROUP, P.O. BOX 14300, WASHINGTON, DC, 20044-4300, US

(21) Appl. No.: 10/520,176

(22) PCT Filed: Aug. 12, 2002
(86) PCT No.: PCT/JP02/08209

§ 371 Date: Jan. 24, 2005

(30) Foreign Application Priority Data
Jul. 11, 2002 (JP) 10231409.8

(51) Int. Cl. A61K 31/198

(52) U.S. Cl. 514/561

(57) ABSTRACT

A method for suppressing proliferation of abnormal prion proteins is provided. Specifically, the method involves systemically, orally, intracerebrally or intraspinally administering an essential amino acid, in particular, one having a branched side chain, that is, one selected from leucine, isoleucine, and valine. Of these, leucine is most preferred.

### TECHNICAL FIELD

The present invention relates to a method for suppressing proliferation of abnormal prion proteins, and more particularly, to a method for suppressing proliferation of abnormal prion proteins through administration of essential amino acids having a branched side chain.

### BACKGROUND ART

Scrapie, the first to be discovered of a group of diseases caused by proliferation of abnormal prion protein, is found in sheep and impairs motor functions of the animal. Scrapie is characterized by porous, sponge-like appearance of affected brains. Other diseases similarly resulting from proliferation of abnormal prion protein were later identified, including bovine spongiform encephalopathy (BSE) in cows, commonly known as mad cow disease, Creutzfeldt-Jakob disease (CJD), and Gerstmann-Straussler-Scheinker syndrome (GSS) in humans.

These diseases were not caused by viral infection, nor had any known pathogen been identified to cause them. However, a specific type of protein commonly found in individuals suffering the disease was suspected to be a pathogenic agent involved in the transmission, or infection, of the disease. The protein was hypothetically named as a prion, or small proteinaceous infectious particle, and the group of diseases caused by the agent has come to be collectively referred to as prion diseases.

Of the known prion diseases, GSS is known to be a familial disease and has proven to be triggered at 100% probability by a point mutation in a prion protein in which one amino acid of the protein is substituted for another type of amino acid (leucine).

The gene encoding prion protein, which consists of 235 amino acids, is located on chromosome 20. The infectious factor of the prion diseases is thought to mostly consist of this prion protein. The type of prion protein that acts as the infectious factor is termed as scrapie-type prion or abnormal prion (PrP^{SC}) while the normal prion is referred to as PrP.

As with the case of GSS, a point mutation in the prion protein can cause onset of other diseases. For example, substitution of proline (Pro) for leucine (Leu) in codon 102 of prion protein triggers cerebellar ataxia, substitution of proline (Pro) for leucine (Leu) in codon 102/219 (Lys) causes dementia or cerebellar ataxia, and substitution of proline (Pro) for leucine (Leu) in codon 105/129 (Val) causes spastic tetraplegia.

Also, in patients suffering fatal familial insomnia (FFI), a prion disease characterized by persistent insomnia and deaths within one year, aspartic acid (Asp) has been substituted for asparagine (Asn) in codon 178 of the prion protein.

Similar diseases caused by mutant prion proteins are also found in various animal species and are observed to transmit from one animal species to another via abnormal prion protein. For example, animal feed contaminated with materials prepared from scrapie-infected sheep has given rise to BSE in cows. Similarly, cat food containing materials prepared from BSE-infected cows has caused feline spongiform encephalopathy (FSE) in cats. Recent studies suggest that there is a significant chance that prion diseases, particularly BSE, are transmitted to human (Collinge J. et. al., Nature 383: 685-690, 1996).

While the manner by which the prion diseases are transmitted via abnormal prion proteins has yet to be clearly understood, it is known that an abnormal prion, despite its nature as protein, retains its infectivity after undergoing sterilizing processes under high pressure or by drying. Furthermore, abnormal prions are not denatured through treatment with formalin, alcohol or phenol. For these reasons, when a person is infected with abnormal prion protein, it is necessary to minimize proliferation of the abnormal prion in order to prevent onset of the disease. However, no effective approach has ever been developed that can suppress the proliferation of abnormal prion protein, nor has any attempt been made to this date to prevent onset of the disease based on such approaches.

### DISCLOSURE OF INVENTION

In view of the present state of the art, it is a primary objective of the present invention to suppress proliferation of abnormal prion protein in animals, including humans, infected with the prion. Also, it is an ultimate goal of the present invention to prevent onset of prion diseases in animals, including humans, infected with the prion.

In an effort to find a way to achieve these objectives, the present inventors have made a finding that proliferation of abnormal prion protein is effectively suppressed by essential amino acids, in particular those having a branched side chain, and thus investigated the possibility of these essential amino acids as a viable suppressive agent for suppressing proliferation of abnormal prion protein. The finding ultimately led the present inventors to bring the present invention to completion.

Accordingly, one essential aspect of the present invention is a method for suppressing proliferation of abnormal prion proteins. The method involves systemically, orally, intracerebrally or intraspinally administering an essential amino acid having a branched side chain at a high concentration.

Specifically, examples of the essential amino acid having a branched side chain for use in the present invention include leucine (Leu), isoleucine (Ile), and valine (Val) and a mixture thereof. The present inventors discovered that, of these amino acids, leucine is particularly effective in suppressing proliferation of abnormal prion protein.

Accordingly, a most preferred embodiment of the present invention is a method for suppressing proliferation of abnormal prion proteins, which involves systemically, orally, intracerebrally or intraspinally administering leucine at a high concentration.

A second essential aspect of the present invention is a method for administering an essential amino acid, involving systemically, orally, intracerebrally or intraspinally administering an essential amino acid having a branched side chain at a high concentration so as to suppress proliferation of abnormal prion proteins.

More specifically, the essential amino acid having a branched side chain to be administered systemically, orally, intracerebrally or intraspinally for the purpose of suppressing proliferation of abnormal prion protein is selected from the group consisting of leucine, isoleucine, valine and a mixture thereof, and most preferably, leucine.

A third essential aspect of the present invention is a suppressive agent for suppressing proliferation of abnormal prion proteins. The suppressive agent contains as an active ingredient an essential amino acid having a branched side chain.

In one specific embodiment, the suppressive agent contains, as the essential amino acid having a branched side chain to serve as an active ingredient, one selected from the group consisting of leucine, isoleucine, valine, and a mixture thereof.

In the most preferred embodiment, the suppressive agent contains leucine as an active ingredient.

A fourth essential aspect of the present invention is a use of an essential amino acid having a branched side chain for being administered systemically, orally, intracerebrally or intraspinally so as to suppress proliferation of abnormal prion proteins.

Specifically, the fourth aspect of the present invention concerns the use of leucine, isoleucine, valine, or a mixture thereof, and most preferably, the use of leucine as the essential amino acid having a branched side chain to be administered systemically, orally, intracerebrally or intraspinally so as to prevent proliferation of abnormal prion protein.

### BRIEF DESCRIPTION OF DRAWING

FIG. 1 is a photograph showing the results of luminescence reactions obtained in Test Example 1 using ECL Western blotting technique.

### BEST MODE CARRYING OUT THE INVENTION

One essential aspect of the present invention is a method for preventing proliferation of an abnormal prion protein through systemic, oral, intracerebral, or intraspinal administration of an essential amino acid having a branched side chain, specifically, one selected from the group consisting of leucine, isoleucine, valine, and a mixture thereof at a high concentration.

The essential amino acid having a branched side chain, or specifically, one selected from the group consisting of leucine, isoleucine and valine, is a safe compound, and the fact that essential amino acids promote growth of human body and help us stay healthy makes the method of the present invention highly effective.

Of the different essential amino acids with a branched side chain, use of leucine has resulted in particularly favorable results. Considering the fact that the infectious, abnormal prion proteins result from a point mutation occurring in the normal prion protein, in particular, substitution of one amino acid for leucine, it is thought that administering leucine can somehow help prevent proliferation of abnormal prion protein.

Thus, in order to suppress proliferation of abnormal prion protein, the essential amino acid having a branched side chain, such as leucine, may be systemically administered through injection. Alternatively, it may be administered orally, intracerebrally or intraspinally. In the latter two cases, the essential amino acid is delivered to brain tissue or spinal cord.

The dosage of the essential amino acid is not limited to a particular range and can be any amount sufficient to suppress proliferation of abnormal prion protein. For example, it may be administered at a large dose of 5 to 15 g/kg. The essential amino acid may be administered as a solution having a high concentration of 20 to 40 mg/mL or, in the case of oral administration, as a solid preparation. In general, the essential amino acid can be administered in the form of a solution with a proper concentration, tablet, powder preparation or other suitable preparations.

### EXAMPLES

The present invention will now be described in detail with reference to the results of a test in which an abnormal prion protein derived from scrapie-infected sheep was used and suppression of proliferation of the protein was observed.

### Test Example 1

Effects that the essential amino acids having a branched side chain have on the proliferation reaction of abnormal prion protein were examined in the following experiment.

1. Method:

Using the protein misfolding cyclic amplification technique (PMCA), abnormal prion protein was amplified In vitro. Brains of sheep collected from a healthy sheep and a scrapie-infected sheep were independently homogenized in 0.5% Triton X-0.05% SDS-PBS (containing a protease inhibitor) to form 10% (w/v) solutions. To each of 10 μL aliquots of the brain homogenate derived from healthy sheep, one-hundredth as much of the brain homogenate derived from scrapie-infected sheep was added to perform PMCA amplification. Leucine was added to a group of the resulting mixtures to a final concentration of 15 mg/mL.

Each mixture of the brain homogenates was incubated for 1 hour while being stirred at 37° C. and was then sonicated using Digital Sonifier 450D available from Branson. Sonication was performed by setting power output to 100% and repeating 5 times a cycle of 0.2-second oscillation period followed by 0.1-second interval.

The mixtures with and without leucine were individually subjected to 0 time (control), 1 time and 10 times of the incubation-sonication treatment to give samples. Proteinase K (PK) was added to each sample to a final concentration of 50 μg/mL, and the samples were incubated for 1 hour at 37° C.

Abnormal prion protein was detected by Western blotting technique. Each sample was separated and electrophoresed by 10% SDS-PAGE and then transferred to a membrane. After blocking nonspecific binding sites on the membrane, each membrane was incubated with rabbit anti-PrP₉₄₋₁₁₂ antibody for 1 hour at room temperature, washed, and then incubated with HRP-labeled anti-rabbit IgG antibody. The membranes were then washed and luminescence was detected by ECL+plus.

2. Results

After PMCA, bands of PK resistant fragments were significantly enhanced in the leucine-free group (e.g., 5-fold in the sample subjected to 10 cycles of the treatment). The degree of enhancement was dependent on the number of cycles.

In contrast, no prominent bands were formed suggesting the presence of PK resistant fragments in the leucine-treated group, including the sample subjected to 10 cycles of PMCA.

Collectively, the results suggest that addition of leucine has effectively suppressed proliferation of abnormal prion protein in vitro.

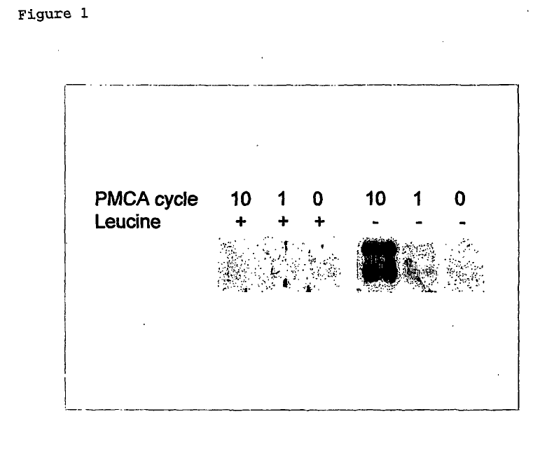

Shown in the FIG. 1 are the results of luminescence reaction performed by ECL+plus. The results obtained for the leucine-treated group are shown on the left of the figure and the results for the leucine-free group on the right.

### INDUSTRIAL APPLICABILITY

As set forth, the method of the present invention to administer essential amino acids having a branched side chain, in particular leucine, can suppress proliferation of abnormal prion proteins.

In view of the fact that no effective measure has been proposed to this date to suppress proliferation of abnormal prion proteins, the suppression of proliferation of abnormal prions, made possible by the present invention, will ultimately lead to prevention of the onset of prion diseases in animals, including humans, infected with an abnormal prion protein and thus proposes a possibility for the treatment of various prion diseases now attracting significant public attention, such as BSE in cows and CJD in humans.

1. A method for suppressing proliferation of abnormal prion proteins, comprising systemically, orally, intracerebrally or intraspinally administering to a patient in need thereof an essential amino acid having a branched side chain.

2. The method according to claim 1, wherein the essential amino acid having a branched side chain is selected from the group consisting of leucine, isoleucine, valine, and mixtures thereof.

3. The method according to claim 2, wherein said amino acid having a branched side chain is leucine.

4. A method for providing an essential amino acid having a branched side chain to a patient in need thereof, comprising systemically, orally, intracerebrally or intraspinally administering said essential amino acid having a branched side chain so as to suppress proliferation of abnormal prion proteins.

5. The method according to claim 4, wherein the essential amino acid having a branched side chain is selected from the group consisting of leucine, isoleucine, valine, and mixtures thereof.

6. The method according to claim 4, wherein said essential amino acid having a branched side chain is leucine.

7. A suppressive agent containing an essential amino acid having a branched side chain as an active ingredient for suppressing proliferation of abnormal prion proteins.

8. The suppressive agent according to claim 7, wherein the essential amino acid having a branched side chain is selected from the group consisting of leucine, isoleucine, valine, and mixtures thereof.

9. The suppressive agent according to claim 8, wherein the essential amino acid having a branched side chain is leucine.

10-12. (canceled)

13. A method for preventing prion diseases by suppressing proliferation of abnormal prion proteins which comprises systemically, orally, intracerebrally or intraspinally administering to a patient in need thereof an essential amino acid having a branched side chain as an active ingredient.

14. The method according to claim 13, wherein the essential amino acid having a branched side chain is selected from the group consisting of leucine, isoleucine, valine, and mixtures thereof.

15. The method according to claim 13, wherein the essential amino acid having a branched side chain is leucine.

16. The method according to claim 13, wherein said prion disease is scrapie.

17. The method according to claim 13, wherein said prion disease is bovine spongiform encephalopathy (BSE).

18. The method according to claim 13, wherein said prion disease is Creutzfeldt-Jakob disease (CJD).

19. The method according to claim 13, wherein said prion disease is Gerstmann-Straussler-Scheinker syndrome.
